(19) United States
(12) Patent Application Publication
(10) Pub. No.: US 2025/0025849 A1
(43) Pub. Date: Jan. 23, 2025

(54) Method for Preparing Natural Starch Microspheres with Uniform Particle Size

(71) Applicant: Ningbo University of Technology, Ningbo (CN)

(72) Inventors: Dan QIU, Ningbo (CN); Zhiguo ZHANG, Ningbo (CN); Raozhen ZUO, Ningbo (CN); Yajuan WANG, Ningbo (CN); Xuechen ZHUANG, Ningbo (CN); Yihui SUN, Ningbo (CN); Jiajia ZHOU, Ningbo (CN)

(73) Assignee: Ningbo University of Technology, Ningbo (CN)

(21) Appl. No.: 18/610,246

(22) Filed: Mar. 19, 2024

(30) Foreign Application Priority Data
Jul. 21, 2023 (CN) 202310902574.7

(51) Int. Cl. B01J 13/12; B01J 13/04

(52) U.S. Cl. CPC B01J 13/12; B01J 13/04

(57) ABSTRACT

The present disclosure provides a method for preparing natural starch microspheres with a uniform particle size. The method includes the following steps: heating natural starch and water for gelatinization to form a water phase; controlling a temperature of a substance including a low-polarity solvent to form an oil phase; dispersing the water phase into the oil phase to obtain a mixed solution; treating the mixed solution under reduced pressure, and carrying out separation to obtain preliminarily solidified starch microspheres; and washing the preliminarily solidified starch microspheres to remove the residual oil phase on the surface, and drying to obtain the natural starch microspheres. Compared with the present technology, the method of the present disclosure can retain the molecular structure of the natural starch and has a high yield, and the prepared natural starch microspheres have a uniform particle size and can load water-soluble and fat-soluble loading objects.

### TECHNICAL FIELD

The present disclosure belongs to the field of natural polymer microspheres, and particularly relates to a method for preparing natural starch microspheres with a uniform particle size.

### BACKGROUND

Starch is the most important edible natural polymer material, which is widely used in many industrial fields such as food, medicine and chemical industry. Natural starch has significant differences in the degree of polymerization of glucose units and the ratio of amylose/amylopectin due to different plant sources, resulting in granules in different shapes and with a particle size of generally greater than 1 μm and up to tens of microns. Natural starch, due to its irregular shapes and wide particle size distribution, has many drawbacks in applications, and thus, needs to be further processed into microspheres with a uniform particle size and regular shapes to meet the needs of multiple fields.

Long chains of glucose in natural starch granules are tightly connect in the form of hydrogen bonds, forming a large number of crystalline regions, so the natural starch granules are insoluble in water. When the starch temperature is higher than the gelatinization temperature and contains a lot of water, the water molecules can enter the microcrystalline interstices of starch, so that the starch irreversibly absorbs a lot of water and the hydrogen bonds between glucose chains are destroyed. The starch granules are transformed into a starch gelatinization solution with high viscosity. In a short gelatinization process, the molecular structure of the starch can be completely retained. Prolonging the gelatinization time, increasing the treatment temperature, or adding acid, alkali or amylase will make the starch irreversibly hydrolyzed, so that some glycosidic bonds are broken to form low molecule saccharides. At the same time, the viscosity of the starch gelatinization solution gradually decreases until the starch is completely dissolved in water. If the gelatinized or hydrolyzed starch is treated at low temperature for a long time, the molecular chains of amylose will undergo irreversible recombination-, so that some starch will retrogradate and solidify and precipitate due to its decrease of solubility. Besides, the molecular chains of starch in the aqueous solution may also be spatially cross-linked by adding a chemical cross-linking agent, so as to reduce the solubility and make the starch solidify and precipitate.

The existing methods for preparing starch microspheres generally include physical method, chemical method, spray-drying method, and inverse emulsion method, etc. The physical method usually grinds and crushes starch granules under the mechanical force. In the physical method, the obtained starch microspheres generally showed large particle size and uneven in dispersion, and moreover, the energy consumption is high. The chemical method refers to hydrolyze starch under alkaline conditions and then mix the hydrolyzed starch with metal ions to produce a precipitate, thus the starch is precipitated and aggregated into microspheres. This method is unable to remove insoluble metal salts, and the degree of hydrolysis of the starch and the shape of the starch microspheres are uncontrollable. In the spray-drying method, the starch is hydrolyzed to form an aqueous solution, and then the aqueous solution is subjected to the shear force of a nozzle under high pressure to form microdroplets; and in a high-temperature environment, water on the surface of the microdroplets quickly evaporate, so that the microspheres are obtained. This method has high requirements for equipment and requires high energy consumption, and the particle size of the microspheres is limited by the size of the nozzle. Moreover, the shape of the microspheres is greatly affected by the drying process, making it difficult to form uniform particle size and regular shapes.

For the inverse emulsion method, the starch is hydrolyzed and then dissolved in water to obtain a water phase, the water phase is dispersed in a surfactant-containing organic phase to form a water-in-oil emulsion; and then a cross-linking agent is added to cross-link the such molecules to form fine microspheres and precipitate. This process can realize loading of the object by adsorption after the microspheres are formed or by dispersing the object in a water phase during the preparation of the microspheres, but the molecular chains of the starch must be sufficiently hydrolyzed to a water-soluble state, so the molecular structure of natural starch cannot be retained. Moreover, the mechanism of action of the cross-linking agent on the molecular chains of the water-soluble starch is complex, making the shapes of the starch microspheres uncontrollable. The loading force between the and starch microspheres and the object is weak, and the cross-linking agent and other residual components contained in the starch microspheres also limit the application of the prepared starch microspheres in the fields of food and medicine, etc. There are many reports on the cross-linking and solidification of molecular chains of starch by introducing cross-linking agents, and the methods and principles are basically similar.

Chinese patent application No. 202010871268.8 disclosed a method for preparing quinoa starch microspheres, including: quinoa starch was hydrolyzed in an NaOH solution and gelatinized to obtain a water phase, then it was added in a soybean oil phase and stirred. The epichlorohydrin was added to the system to cross-link the starch molecular chains, and the 10-35 mm starch microspheres was obtained after the centrifugation. In this method, the natural starch was hydrolyzeda certain extent, the chemical cross-linking agent was introduced, and it was difficult to make the particle size uniform.

In addition to the above-mentioned representative technologies, there are some other methods for preparing starch microspheres, but it is still not possible to control the particle size and shape of the microspheres, and the uniformity of and the yield of the prepared starch microspheres were low.

For example, Chinese patent application No. 201010546156.1 disclosed a method for preparing starch microspheres, including: a starch granule dispersion and a thickener solution were treated at the critical temperature of starch gelatinization for 10 to 30 minutes, and the obtained solution was allowed to stand and cooled to obtain the starch hollow microspheres with a particle size of 10-100 nm. The thickener was used as the core, and the starch molecules were slightly gelatinized and then recrystallized so as to be coated outside the thickener. However, the thickener was a necessary core additive, and the particle size and the shape of the microspheres were difficult to control, making it impossible to achieve a uniform particle size.

Chinese patent application No. 201510151917.6 disclosed a method for preparing nano-starch microspheres, including: amylose was added to an alkaline solution to form a suspension, and the suspension was frozen, melted at a higher temperature, dialyzed and filtered to obtain the nano-starch microspheres. In this method, based on the characteristic of high tendency to retrogradation of the amylose at low temperature, some molecular chains of the amylose are recombined so as to solidify and precipitate to form the microspheres. However, strict debranching of natural starch was required, the yield of the starch microspheres was not high, and the particle size distribution was still wide.

Chinese patent application No. 201710108480.7 disclosed a method for preparing starch microspheres with a small particle size, including: acid-hydrolyzed natural starch was gelatinized, mixed with a polyvinylpyrrolidone solution and emulsified, the emulsion was retrogradated at low temperature for 24 h, and separation was carried out to obtain 1-10 mm starch microspheres. This method needed to hydrolyze the natural starch in advance, and the solidified starch microspheres obtained by retrogradation were not high in yield and not uniform in particle size.

Chinese patent application No. 201710430433.4 disclosed a method for preparing starch crystalline microspheres, including: starch after enzymatic hydrolysis, acid hydrolysis and freeze-drying was grounded and crushed, and then gelatinized and retrogradated at low-temperature overnight, and separation was carried out to obtain 1-5 mm starch crystalline microspheres. This method required complex pretreatment of natural starch. The uniformity of particle size was improved, but the yield was low.

Chinese patent application No. 201710694631.1 disclosed a method for preparing starch microspheres based on a two-phase aqueous system, including: natural starch was acid-hydrolyzed and gelatinized, and then mixed with a polyethylene glycol solution to form a two-phase system aqueous emulsion; and then, the two-phase aqueous system emulsion was treated at low temperature for 4-24 h, and centrifugation was carried out to obtain the 0.1-30 mm starch microspheres formed by retrogradation. This method was basically consistent with the paper “Effect of molecular weight of starch on the properties of cassava starch microspheres prepared in aqueous two-phase system” (Carbohydrate Polymers, 2017), which also required hydrolysis of natural starch and obtained starch microspheres with nonuniform particle size.

In addition, “Preparation of tailor-made starch-based aerogel microspheres by the emulsion gelation method” (Carbohydrate Polymers, 2012) disclosed a method for preparing starch microspheres, in this paper, based on the emulsion gelation method, natural starch was dispersed in water and mixed with vegetable oil then emulsified, and the emulsion was heated to 368-413 K under pressure for 20 min; then emulsion was cooled to 318 K at a speed of 3 K/min in an ice water bath, and centrifugation was carried out to obtain preliminarily solidified microspheres; and the preliminarily solidified microspheres retrogradated in a refrigerator for 48 h, mixed with ethanol, and dried by supercritical carbon dioxide to obtain the 215-1226 mm starch microspheres. In this method, the starch granules in the emulsion was partially gelatinized/gelated, retrogradation during cooling process and supercritical drying to obtain the microspheres. The treatment process was complicated, the molecular structure of the starch was changed to some extent, and the uniformity of particle size of the microspheres was still not ideal.

Based on the above, the existing methods generally use the suspended dispersion of starch granules or the gelatinization solution of hydrolyzed starch as the water phase, and there is no report on the use of the natural starch gelatinization solution as the water phase. This is because although the molecular structure of the natural starch gelatinization solution heated for a short time can be completely retained, the gelatinization solution generally has a high viscosity. Once the temperature is lower than the gelatinization temperature, the gelatinization solution will quickly be transformed into a discontinuous complex colloid, which will quickly coagulate and be difficult to disperse when added to another water phase or oil phase.

In addition, in the existing methods, the molecular chains of starch are recombined and solidified by the introduction of chemical cross-linking agents or low-temperature retrogradation, but the proportion of starch solidified and the yield of microspheres are not high; and moreover, it is difficult to control the degree of recombination of the molecular chains of starch, thus making it difficult to make the particle size uniform. Some methods using chemical cross-linking agents to load object and realize loading during cross-linking and microsphere formation, and there is still no report on other methods without using chemical cross-linking agents that can realize effective loading during the preparation of microspheres.

### SUMMARY

In order to solve the technical problems in the present technology, the invention is directed to a method for preparing natural starch microspheres with a high yield and a uniform particle size while retaining the molecular structure of natural starch.

In view of the above technical problems, the invention provides a method for preparing natural starch microspheres with a uniform particle size, including the following steps:
S1: heating a mixture of natural starch and water for gelatinization to form a water phase;
S2: controlling a temperature of a substance including a low-polarity solvent to form an oil phase;
S3: dispersing the water phase obtained in step S1 into the oil phase obtained in step S2 to obtain a mixed solution;
S4: treating the mixed solution obtained in step S3 under reduced pressure, and separation to obtain preliminarily solidified starch microspheres; and
S5: washing the preliminarily solidified starch microspheres obtained in step S4 to remove the residual oil phase on the surface of the preliminarily solidified starch microspheres, and drying to obtain the natural starch microspheres.

The natural starch in step S1 includes, but not limited to, corn starch, waxy corn starch, bean starch, potato starch, sweet potato starch, wheat starch, cassava starch, grain amaranth starch, rice starch, water caltrop starch, lotus root starch, kudzu root starch and water chestnut starch.

Preferably, the natural starch in step S1 is at least one of corn starch, waxy corn starch, bean starch, potato starch, sweet potato starch, wheat starch, cassava starch, grain amaranth starch, rice starch, water caltrop starch, lotus root starch, kudzu root starch and water chestnut starch.

Further preferably, the natural starch in step S1 is at least one of corn starch, waxy corn starch, mung bean starch, pea starch, potato starch, sweet potato starch and cassava starch.

Further preferably, the natural starch in step S1 is at least one of corn starch, waxy corn starch, mung bean starch, pea starch, potato starch, sweet potato starch, cassava starch and grain amaranth starch.

As a specific example of the present disclosure, the natural starch in step S1 is corn starch.

As a specific example of the present disclosure, the natural starch in step S1 is cassava starch.

As a specific example of the present disclosure, the natural starch in step S1 is waxy corn starch.

As a specific example of the present disclosure, the natural starch in step S1 is grain amaranth starch.

Preferably, the starch and the water in step S1 are respectively used in an amount of 1 part by mass and 6-100 parts by mass.

Further preferably, the starch and the water in step S1 are respectively used in an amount of 1 part by mass and 9-50 parts by mass.

Preferably, the gelatinization in step S1 is carried out at a temperature of 50-100° C. for 1-300 min.

Further preferably, the gelatinization in step S1 is carried out at a temperature of 75-100° C. for 3-30 min.

As a specific example of the present disclosure, the gelatinization in step S1 is carried out at a temperature of 100° C. for 30 min.

As a specific example of the present disclosure, the gelatinization in step S1 is carried out at a temperature of 85° C. for 8 min.

As a specific example of the present disclosure, the gelatinization in step S1 is carried out at a temperature of 90° C. for 7 min.

Further preferably, the gelatinization in step S1 is carried out at a temperature of 75-80° C. for 3-5 min.

As a specific example of the present disclosure, the gelatinization in step S1 is carried out at a temperature of 75° C. for 3 min.

As a specific example of the present disclosure, the gelatinization in step S1 is carried out at a temperature of 80° C. for 3 min.

As a specific example of the present disclosure, the gelatinization in step S1 is carried out at a temperature of 80° C. for 5 min.

Preferably, the water phase in step S1 has a viscosity of 1.1-1000 cP.

As a specific example of the present disclosure, the water phase in step S1 has a viscosity of 1000 cP.

As a specific example of the present disclosure, the water phase in step S1 has a viscosity of 1.1 cP.

Further preferably, the water phase in step S1 has a viscosity of 3-500 cP.

Further preferably, the water phase in step S1 has a viscosity of 3.4-436 cP.

As a specific example of the present disclosure, the water phase in step S1 has a viscosity of 436 cP.

As a specific example of the present disclosure, the water phase in step S1 has a viscosity of 10 cP.

As a specific example of the present disclosure, the water phase in step S1 has a viscosity of 100 cP.

As a specific example of the present disclosure, the water phase in step S1 has a viscosity of 3.4 cP.

Preferably, the low-polarity solvent in step S2 is at least one of aliphatic hydrocarbon, vegetable oil, animal oil, beeswax and MCT. The MCT is medium chain triglyceride, the same below.

As a specific example of the present disclosure, the low-polarity solvent in step S2 is cocoa butter.

As a specific example of the present disclosure, the low-polarity solvent in step S2 is soybean oil.

As a specific example of the present disclosure, the low-polarity solvent in step S2 is lard.

Further preferably, the low-polarity solvent in step S2 is at least one of aliphatic hydrocarbon, beeswax and MCT.

Further preferably, the low-polarity solvent in step S2 is one of aliphatic hydrocarbon, beeswax and MCT.

As a specific example of the present disclosure, the low-polarity solvent in step S2 is MCT.

As a specific example of the present disclosure, the low-polarity solvent in step S2 is beeswax.

As a specific example of the present disclosure, the low-polarity solvent in step S2 is n-hexane.

As a specific example of the present disclosure, the low-polarity solvent in step S2 is n-octane.

Preferably, the oil phase in step S2 includes an oil phase surfactant.

Further preferably, the surfactant is selected from at least one of Tween 80 and Span 80.

More preferably, the surfactant is selected from one of Tween 80 and Span 80.

Further preferably, in step S2, a surfactant accounting for 0.1-2% by mass of the oil phase is added.

As a specific example of the present disclosure, the surfactant in step S2 is Span 80, and accounts for 1% by mass of the oil phase.

As a specific example of the present disclosure, the surfactant in step S2 is Tween 80, and accounts for 2% by mass of the oil phase.

Preferably, the temperature in step S2 is 0-100° C.

As a specific example of the present disclosure, the temperature in step S2 is 0° C.

As a specific example of the present disclosure, the temperature in step S2 is 100° C. Further preferably, the temperature in step S2 is 30-80° C.

As a specific example of the present disclosure, the temperature in step S2 is 30° C.

As a specific example of the present disclosure, the temperature in step S2 is 40° C.

As a specific example of the present disclosure, the temperature in step S2 is 80° C.

As a specific example of the present disclosure, the temperature in step S2 is 50° C.

The dispersion in step S3 includes, but not limited to, at least one of a manual dropping process, a membrane emulsification process, a high-voltage electrostatic control process and a microfluidic process.

Preferably, the dispersion in step S3 is at least one of a manual dropping process, a membrane emulsification process, a high-voltage electrostatic control process and a microfluidic process.

More preferably, the dispersion in step S3 is one of a manual dropping process, a membrane emulsification process, a high-voltage electrostatic control process and a microfluidic process.

More preferably, the dispersion in step S3 is a manual dropping process or a membrane emulsification process.

As a specific example of the present disclosure, the dispersion in step S3 is a manual dropping process.

As a specific example of the present disclosure, the dispersion in step S3 is a membrane emulsification process.

More preferably, the dispersion used in step S3 is a manual dropping process, and specifically includes the following steps:

the water phase obtained in step S1 is manually dropped into the oil phase obtained in step S2 with a syringe, and the water phase is dropped in different positions to form droplets that do not adhere to each other, thereby obtaining the mixed solution.

Further preferably, a needle of the syringe has an inner diameter of 0.1-1 mm.

Further preferably, a needle of the syringe has an inner diameter of 0.21-0.41 mm.

As a specific example of the present disclosure, a needle of the syringe has an inner diameter of 0.21 mm. During this process, the initial droplets generated have an average particle size of 0.9 mm.

As a specific example of the present disclosure, a needle of the syringe has an inner diameter of 0.41 mm. During this process, the initial droplets generated have an average particle size of 1.1 mm.

Preferably, the oil phase is not stirred during the manual dropping.

Preferably, in the manual dropping process, the water phase obtained in step S1 has a viscosity of 100-1000 cp.

As a specific example of the present disclosure, in the manual dropping process, the water phase obtained in step S1 has a viscosity of 436 cp.

As a specific example of the present disclosure, in the manual dropping process, the water phase obtained in step S1 has a viscosity of 1000 cp.

As a specific example of the present disclosure, in the manual dropping process, the water phase obtained in step S1 has a viscosity of 100 cp.

Preferably, the dispersion used in step S3 is a membrane emulsification process, and specifically includes the following steps:

the water phase obtained in step S1 is passed through a hydrophobic membrane with a pore size of 0.1-50 μm under a transmembrane pressure of 0.01-0.5 MPa, and the filtrate is dispersed into the oil phase obtained in step S2 to form the mixed solution.

As a specific example of the present disclosure, in the membrane emulsification process, the transmembrane pressure is 0.5 MPa, and the pore size of the hydrophobic membrane is 50 μm.

More preferably, the transmembrane pressure is 0.04-0.4 MPa, and the pore size of the hydrophobic membrane is 0.1-10 μm.

As a specific example of the present disclosure, in the membrane emulsification process, the transmembrane pressure is 0.04 MPa, and the pore size of the hydrophobic membrane is 0.1 μm.

Preferably, in the membrane emulsification process, the oil phase is stirred.

More preferably, the stirring is carried out at a speed of 10-700 rpm.

As a specific example of the present disclosure, the stirring is carried out at a speed of 400 rpm.

More preferably, in the membrane emulsification process, the water phase in step S1 has a viscosity of 1.1-10 cp.

As a specific example of the present disclosure, in the membrane emulsification process, the water phase in step S1 has a viscosity of 3.4 cP.

As a specific example of the present disclosure, in the membrane emulsification process, the water phase in step S1 has a viscosity of 1.1 cP.

As a specific example of the present disclosure, in the membrane emulsification process, the water phase in step S1 has a viscosity of 10 cP.

As a specific example of the present disclosure, the dispersion in step S3 is a microfluidic process, and specifically includes the steps as follows.

The water phase in step S1 and the oil phase in step S2 are respectively added to corresponding liquid storage tubes, and a PDMS/glass bonded microfluidic chip with a channel width of 80-350 μm and a nozzle width of 60 μm is used to produce the microspheres at a water phase flow rate of 15 L/min and an oil phase flow rate of 35 μL/min. The initial droplets generated have an average particle size of 100 μm. Thus, a W/O emulsion is obtained.

As a specific example of the present disclosure, the dispersion in step S3 is a high-voltage electrostatic control process, and specifically includes the steps as follows.

A bottom of the container filled with the oil phase in step S2 and the needle of the syringe are respectively connected to positive and negative electrodes of a high-voltage electrostatic droplet generator, and the water phase is slowly injected into the oil phase through the needle of the syringe under the action of a micro-injection pump. The needle of the syringe has an inner diameter of 0.21 mm, the voltage is 6 kV, the push speed is 10 mm per hour, and the receiving distance is 20 mm. Initial droplets with an average particle size of 0.4 mm are formed, thereby obtaining a W/O emulsion.

Preferably, the reduced pressure treatment in step S4 is carried out at a temperature of 40-100° C. under an atmospheric pressure of 1-200 mmHg for 15-600 min.

Further preferably, the reduced pressure treatment in step S4 is carried out at a temperature of 45 to 90° C. under an atmospheric pressure of 1 to 100 mmHg for 15 to 300 min.

More preferably, the reduced pressure treatment in step S4 is carried out at a temperature of 60-80° C. under an atmospheric pressure of 3-50 mmHg for 15-120 min.

As a specific example of the present disclosure, the reduced pressure treatment in step S4 is carried out at a temperature of 80° C. under an atmospheric pressure of 3 mmHg for 120 min.

As a specific example of the present disclosure, the reduced pressure treatment in step S4 is carried out at a temperature of 60° C. under an atmospheric pressure of 3 mmHg for 120 min.

As a specific example of the present disclosure, the reduced pressure treatment in step S4 is carried out at a temperature of 80° C. under an atmospheric pressure of 50 mmHg for 120 min.

As a specific example of the present disclosure, the reduced pressure treatment in step S4 is carried out at a temperature of 80° C. under an atmospheric pressure of 3 mmHg for 15 min.

Preferably, in step S4, the separation is at least one of centrifugation and filtration.

Further preferably, in step S4, the separation is centrifugation.

Preferably, the washing in step S5 is washing with an organic solvent.

Further preferably, in step S5, the solvent used for the washing is at least one of alcohols, ketones, hydrocarbons, esters and halohydrocarbons.

More preferably, in step S5, the solvent used for the washing is at least one of methanol, ethanol, acetone, 2-butanone, cyclohexane, ethyl acetate and dichloromethane.

As a specific example of the present disclosure, in step S5, the solvent used for the washing is ethanol.

As a specific example of the present disclosure, in step S5, the solvent used for the washing is cyclohexane.

Preferably, in step S5, the drying is one of freeze-drying or oven blast drying.

Preferably, the drying is carried out for 3 to 12 h.

As a specific example of the present disclosure, in step S5, the drying is oven blast drying at 40° C. for 12 h.

Further preferably, the drying is freeze-drying for 3-12 h.

As a specific example of the present disclosure, in step S5, the drying is freeze-drying for 10 h.

As a specific example of the present disclosure, in step S5, the drying is freeze-drying for 8 h.

The present disclosure further provides a method for preparing a natural starch microsphere carrier, which further includes the following step on the basis of the method for preparing natural starch microspheres with a uniform particle size:

additionally, adding at least one of a water-soluble loading object, a water-soluble colloid and a fat-soluble loading object into the water phase in step S1.

The water-soluble loading object includes, but not limited to, vitamin C, vitamin B2, anthocyanin, folic acid, etc.

Preferably, the water-soluble loading object is selected from at least one of vitamin C, vitamin B2, anthocyanin and folic acid. The water-soluble loading object is used in an amount of 0.01-1 part by mass.

As a specific example of the present disclosure, the water-soluble loading object is vitamin C.

The water-soluble colloid includes, but not limited to, unhydrolyzed starch octenyl succinate, hydrolyzed starch octenyl succinate, gelatin, gum arabic, carboxymethyl cellulose, lignosulfonate, guar gum, xanthan gum, etc.

Preferably, the water-soluble colloid is selected from at least one of unhydrolyzed starch octenyl succinate, hydrolyzed starch octenyl succinate, gelatin, gum arabic, sodium carboxymethyl cellulose, sodium lignosulfonate, calcium lignosulfonate, guar gum and xanthan gum. The water-soluble colloid is used in an amount of 0-1 part by mass.

As a specific example of the present disclosure, the water-soluble colloid is xanthan gum.

The fat-soluble loading object includes, but not limited to, vitamin A, vitamin E, paclitaxel, β-carotene, etc. The state of the fat-soluble loading object includes, but not limited to, a solid state, a saturated solution in ethanol, etc.

Preferably, the fat-soluble loading object is at least one of vitamin A, vitamin E, paclitaxel, β-carotene, etc.

More preferably, the fat-soluble loading object is one of vitamin A, vitamin E, paclitaxel, β-carotene, etc. The fat-soluble loading object is used in an amount of 0.01-1 part by mass.

As a specific example of the present disclosure, the fat-soluble loading object is vitamin E.

As a specific example of the present disclosure, the fat-soluble loading object is paclitaxel.

As a specific example of the present disclosure, the fat-soluble loading object is β-carotene.

Preferably, the water-soluble colloid, the water-soluble loading object and the fat-soluble loading object is added to the water phase in step S1 after the gelatinization is completed.

Compared with the present technology, the present disclosure has the following beneficial effects:

(1) According to the method provided by the present disclosure, through natural starch gelatinization, droplet dispersion and vacuum dehydration, hydrolysis and cross-linking of the molecular chains of starch are avoided, so that the starch in the prepared starch microspheres does not undergo obvious retrogradation, and thus, the molecular chains of natural starch are completely retained.

(2) The method provided by the present disclosure is simple to operate, and the prepared microspheres have a uniform and controllable average particle size, regular shapes, and a yield greater than 95%.

(3) The microspheres prepared by this method can effectively load different water-soluble and fat-soluble loading objects.

(4) The microspheres prepared by this method contain only one component, and are convenient to process and applicable to food, medicine and other fields.

### BRIEF DESCRIPTION OF FIGURES

FIG. 1 shows electron micrographs of starch and starch microspheres, where a is a scanning electron micrograph of commercially available corn starch, b is a scanning electron micrograph of starch microspheres prepared in Example 1, and c is a scanning electron micrograph of starch microspheres prepared in Example 3;

FIG. 2 is a DSC curve of corn starch, starch microspheres and starch microsphere carrier, where a represents the corn starch, b represents the starch microspheres prepared by the method in Example 1, and c represents the starch microsphere carrier prepared by the method in Example 15; and

FIG. 3 shows XRD patterns of corn starch, starch microspheres and starch microsphere carrier, where a represents the corn starch, b represents the starch microspheres prepared by the method in Example 1, and c represents the starch microsphere carrier prepared by the method in Example 15.

### DETAILED DESCRIPTION

The following non-limiting examples can make those of ordinary skill in the technology to further understand the present disclosure, but do not limit the present disclosure in any form. The following contents are merely exemplary descriptions of the scope of the present disclosure, and those skilled in the technology can make various changes and modifications to the present disclosure according to the disclosed contents, which shall also fall into to the scope of the present disclosure.

The present disclosure will be further described below in conjunction with the specific examples. Unless otherwise specified, various chemical reagents used in the examples of the present disclosure are obtained by conventional commercial means. Unless otherwise specified, the contents mentioned below are all mass contents. Unless otherwise specified, it is understood to be carried out at room temperature.

The examples and comparative examples of the method for preparing natural starch microspheres with a uniform particle size specifically includes the steps as follows.

### Example 1

S1: 1 g corn starch and 9 g water are gelatinized at 75° C. for 3 min to form a water phase with a viscosity of 436 cP.

S2: A temperature of MCT is adjusted to 0° C. to form an oil phase.

S3: Without stirring, the water phase is manually dropped and dispersed into the oil phase obtained in step S2 with a syringe (whose needle has an inner diameter of 0.21 mm) to form initial droplets with an average particle size of 0.9 mm, thereby obtaining a mixed system.

S4: The mixed system obtained in step S3 is treated at 80° C. under an atmospheric pressure of 3 mmHg for 120 min and then centrifuged, and the supernatant is discarded so as to obtain preliminarily solidified starch microspheres.

S5: The starch microspheres are washed with ethanol to remove the residual oil phase on the surface, and then freeze-dried for 8 h to obtain the dried starch microspheres.

### Example 2

S1: 1 g cassava starch and 6 g water are gelatinized at 80° C. for 3 min to form a water phase with a viscosity of 1000 cP.

S2: Cocoa butter is heated to 50° C., followed by the addition of 1% Span 80. The mixture is stirred uniformly to form an oil phase.

S3: Without stirring, the water phase is manually dropped and dispersed into the oil phase obtained in step S2 with a syringe (whose needle has an inner diameter of 0.41 mm) to form initial droplets with an average particle size of 1.1 mm, thereby obtaining a mixed system.

S4: The mixed system obtained in step S3 is treated at 60° C. under an atmospheric pressure of 3 mmHg for 120 min and then centrifuged, and the supernatant is discarded so as to obtain preliminarily solidified starch microspheres.

S5: The starch microspheres are washed with cyclohexane to remove the residual oil phase on the surface, and then freeze-dried for 10 h to obtain the dried starch microspheres.

### Example 3

S1: 5 g corn starch and 95 g water are gelatinized at 100° C. for 30 min to form a water phase with a viscosity of 3.4 cP.

S2: Lard is heated to 100° C., followed by the addition of 2% Tween 80. The mixture is stirred uniformly to form an oil phase.

S3: With stirring at 400 rpm and under a pressure of 0.04 Mpa, the water phase is dispersed into the oil phase obtained in step S2 through a hydrophobic microporous membrane (with a pore size of 0.1 μm) of an SPG membrane emulsifier to form initial droplets with an average particle size of 0.12 μm, thereby obtaining a W/O emulsion.

S4: The mixed solution obtained in step S3 is treated at 80° C. under an atmospheric pressure of 50 mmHg for 120 min, and then centrifuged to obtain preliminarily solidified starch microspheres.

S5: The starch microspheres are washed with ethanol to remove the residual oil phase on the surface, and then blast-dried in an oven at 40° C. for 12 h to obtain the dried starch microspheres.

### Example 4

Compared with Example 1, in step S2, beeswax is heated to 80° C. to form the oil phase, and the rests are the same.

### Example 5

Compared with Example 1, in step S2, n-hexane is heated as the low-polarity solvent to 30° C. to form the oil phase, and the rests are the same.

### Example 6

Compared with Example 1, in step S2, n-octane is heated as the low-polarity solvent to 40° C. to form the oil phase, and the rests are the same.

### Example 7

Compared with Example 3, in step S3, the dispersion method is a microfluidic process, and the rests are the same.

The microfluidic process specifically includes the following steps:

The water phase in step S1 and the oil phase in step S2 are respectively added to corresponding liquid storage tubes, and a PDMS/glass bonded microfluidic chip with a channel width of 80-350 μm and a nozzle width of 60 μm is used to produce the microspheres at a water phase flow rate of 15 μL/min and an oil phase flow rate of 35 L/min. The initial droplets generated have an average particle size of 100 μm. Thus, a W/O emulsion is obtained.

### Example 8

Compared with Example 3, in step S3, the dispersion is a high-voltage electrostatic control process, and the rests are the same.

The high-voltage electrostatic control process specifically includes the following steps:

A bottom of the container filled with the oil phase in step S2 and the needle of the syringe are respectively connected to positive and negative electrodes of a high-voltage electrostatic droplet generator, and the water phase is slowly injected into the oil phase through the needle of the syringe under the action of a micro-injection pump. The needle of the syringe has an inner diameter of 0.21 mm, the voltage is 6 kV, the push speed is 10 mm per hour, and the receiving distance is 20 mm. Initial droplets with an average particle size of 0.4 mm are formed, thereby obtaining a W/O emulsion.

### Example 9

Compared with Example 1, in step S1, 1 g corn starch and 13 g water are gelatinized at 80° C. for 5 min to form a water phase with a viscosity of 100 cP, and the rests are the same.

### Example 10

Compared with Example 3, in step S1, 0.1 g waxy corn starch and 10 g water are gelatinized at 85° C. for 8 min to form a water phase with a viscosity of 1.1 cP, and the rests are the same.

### Example 11

Compared with Example 3, in step S1, 0.5 g grain amaranth starch and 7.5 g water are gelatinized at 90° C. for 7 min to form a water phase with a viscosity of 10 cP, the pore size of the microporous membrane is 50 μm, and the transmembrane pressure is 0.5 MPa. The rests are the same.

### Example 12

Compared with Example 1, in step S4, the reduced pressure treatment is carried out for 15 min, and the rests are the same.

### Example 13

Compared with Example 1, in step S2, soybean oil is used, and the rests are the same.

### Example 14

Compared with Example 3, in step S2, the amount of the Tween 80 added is 0.1%. Step S3 specifically includes:

With stirring at 400 rpm and under a pressure of 0.4 Mpa, the water phase is dispersed into the oil phase obtained in step S2 through a hydrophobic microporous membrane (with a pore size of 10 μm) of an SPG membrane emulsifier to form initial droplets with an average particle size of 11.4 μm, thereby obtaining a W/O emulsion.

The rests are the same.

### Comparative Example 1

Compared with Example 1, in step S1, 1 g corn starch and 7.3 g water are gelatinized at 80° C. for 5 min to form a water phase with a viscosity of 1500 cP, and the rests are the same.

### Comparative Example 2

Compared with Example 1, in step S3, the water phase obtained in step S1 is added to the oil phase obtained in step S2 under the shear emulsification action of 1000 rpm to form a mixture, and the rests are the same.

### Comparative Example 3

Compared with Example 1, there is no step S4, and the rests are the same.

### Comparative Example 4

Compared with Example 1, the treatment under reduced pressure in step S4 is carried out under the conditions below, and the rests are the same:

“the temperature is 90° C., the atmospheric pressure is 60 mmHg, and the time is 150 min.”

The particle size and yield of natural starch microspheres prepared according to the methods described in the examples and the comparative examples are shown in the table below.

Yield by

| | | |
|---|---|---|
| No. | Average Particle Size* | Mass |

| | | | |
|---|---|---|---|
| Example 1 | 1.22 ± 0.05 | mm | 99% |
| Example 2 | 1.53 ± 0.04 | mm | 99% |
| Example 3 | 1.73 ± 0.02 | μm | 95% |
| Example 4 | 1.20 ± 0.01 | mm | 99% |
| Example 5 | 1.21 ± 0.03 | mm | 99% |
| Example 6 | 1.21 ± 0.03 | mm | 99% |
| Example 7 | 134.35 ± 14.92 | μm | 96% |
| Example 8 | 0.95 ± 0.11 | mm | 97% |
| Example 9 | 1.16 ± 0.11 | mm | 96% |
| Example 10 | 1.47 ± 0.01 | μm | 95% |
| Example 11 | 63.12 ± 2.03 | μm | 96% |
| Example 12 | 1.24 ± 0.09 | mm | 98% |
| Example 13 | 1.22 ± 0.04 | mm | 99% |
| Example 14 | 12.32 ± 0.72 | mm | 94% |
| Comparative | 1.34 ± 0.25 | mm | 34% |
| Example 1 | | | |

| | | |
|---|---|---|
| Comparative | The microspheres cannot | 0 |
| Example 2 | be formed | |
| Comparative | The droplets coalesce and | 0 |
| Example 3 | the microspheres cannot | |
| | be formed | |
| Comparative | The dehydration and | 0 |
| Example 4 | solidification under | |
| | reduced pressure are not | |
| | effective, the droplets | |
| | coalesce and are | |
| | deformed, and the | |
| | microspheres cannot be | |
| | formed | |

*The average particle size of the microspheres given in the above table is the average value and standard deviation of particle sizes (the maximum diameter is recorded as the particle size) of more than 10 microspheres measured on a microscope scale.

As can be seen from the data in the above table:

Compared with Example 1, in Comparative Example 1, due to the excessive viscosity of the starch gelatinization solution, the droplets are discontinuous, the shapes of the microspheres are irregular, and the yield is severely reduced.

Compared with Example 1, in Comparative Example 2, after the water phase in step S1 and the oil phase in step S2 are mixed under the shear emulsification action, the formation of the disperse system is complex, and the starch gelatinization solution is irregularly dispersed in the oil phase, so that the droplets and the subsequent microspheres cannot be formed.

Compared with Example 1, in Comparative Example 3, the formed droplets are not dehydrated under reduced pressure in time, resulting in collision and coalescence of the droplets, so that the microspheres cannot be formed.

Compared with Example 1, in Comparative Example 4, due to the insufficient vacuum during the treatment in step S4, the droplets are not effectively dehydrated under reduced pressure, and the droplets with high water content directly coalesce and are deformed during subsequent filtration, so that the microspheres cannot be formed.

The examples and comparative examples of the method for preparing a natural starch microsphere carrier specifically includes the steps as follows.

### Example 15

S1: 1 g corn starch and 9 g water are gelatinized at 80° C. for 3 min, and then, 1 g vitamin C is added to form a water phase with a viscosity of 483 cP.

S2: MCT is heated to 0° C. to form an oil phase.

S3: Without stirring, the water phase is manually dropped and dispersed into the oil phase obtained in step S2 with a syringe (whose needle has an inner diameter of 0.21 mm) to form initial droplets with an average particle size of 0.85 mm, thereby obtaining a mixed solution.

S4: The mixed solution obtained in step S3 is treated at 50° C. under an atmospheric pressure of 50 mmHg for 120 min, and then centrifuged to obtain preliminarily solidified starch microspheres.

S5: The starch microspheres are washed with ethanol to remove the residual oil phase on the surface, and then freeze-dried for 8 h to obtain the dried starch microspheres.

### Example 16

S1: 1 g cassava starch and 9 g water are gelatinized at 75° C. for 3 min, and then, 0.5 g vitamin C and 0.5 g vitamin E are added to form a water phase with a viscosity of 427 cP.

S2: MCT is heated to 0° C. to form an oil phase.

S3: Without stirring, the water phase is manually dropped and dispersed into the oil phase obtained in step S2 with a syringe (whose needle has an inner diameter of 0.21 mm) to form initial droplets with an average particle size of 0.78 mm, thereby obtaining a mixed system.

S4: The mixed solution obtained in step S3 is treated at 50° C. under an atmospheric pressure of 50 mmHg for 120 min, and then centrifuged to obtain preliminarily solidified starch microspheres.

S5: The starch microspheres are washed with ethanol to remove the residual oil phase on the surface, and then freeze-dried for 8 h to obtain the dried starch microspheres.

### Example 17

Compared with Example 16, there is no water-soluble loading object, and the fat-soluble loading object is vitamin E in an amount of 1 g. The rests are the same.

### Example 18

Compared with Example 16, there is no water-soluble loading object, and the fat-soluble loading object is β-carotene in an amount of 0.02 g, which is dissolved in 10 g of ethanol.

The rests are the same.

### Example 19

Compared with Example 16, there is no water-soluble loading object, the fat-soluble loading object is paclitaxel in an amount of 0.32 g, which is dissolved in 10 g of ethanol, and further, there is a water-soluble carrier xanthan gum in an amount of 0.5 g. The rests are the same.

### Comparative Example 5

Compared with Comparative Example 2, in step S1, before the gelatinization, 1 g vitamin C is also added to the mixture. The rests are the same.

### Comparative Example 6

Compared with Comparative Example 3, in step S1, before the gelatinization, 1 g vitamin C is also added to the mixture. The rest is the same.

### Comparative Example 7

Compared with Example 19, no water-soluble carrier is added, and the rests are the same.

The results of load experiments prepared in Examples 15-19 and Comparative Examples 5-7 are shown in the table below.

| | Loading | Drug Loading | Encapsulation |
|---|---|---|---|
| Example No. | Object | amount (%) | Efficiency (%) |
| Example 15 | Vitamin C | 32.2 | 64.4 |
| Example 16 | Vitamin C | 13.7 | 54.8 |
| | Vitamin E | 14.3 | 57.2 |
| Example 17 | Vitamin E | 31.9 | 63.7 |
| Example 18 | B-carotene | 1.47 | 75.0 |
| Example 19 | Paclitaxel | 14.6 | 83.2 |
| Comparative | Vitamin C | The microspheres cannot be formed, and the | |
| Example 5 | | drug loading amount cannot be determined | |
| Comparative | Vitamin C | The microspheres cannot be formed, and the | |
| Example 6 | | drug loading amount cannot be determined | |
| Comparative | Paclitaxel | 18.9 | 77.9 |
| Example 7 | | | |

As can be seen from the above table:

In Comparative Example 5, after the water phase in step S1 and the oil phase in step S2 are mixed under the emulsification, since the homogeneous phase cannot be formed, the microspheres cannot be formed, and the vitamin C cannot be loaded, which is similar to Comparative Example 2.

In Comparative Example 6, no reduced pressure treatment is carried out. Similar to Comparative Example 3, the formed droplets are not dehydrated under reduced pressure in time, resulting in collision and coalescence of the droplets, so that the microspheres cannot be formed, and the vitamin C cannot be loaded.

Compared with Example 19, in Comparative Example 7, since no water-soluble carrier is used, the drug loading amount is increased, but the encapsulation efficiency is significantly reduced, which indicates that the addition of the water-soluble carrier helps in better loading and protecting the fat-soluble loading object.

The following experiments further characterize the preparation method described herein, and the starch microspheres prepared by the preparation method of the invention.

The shapes of the commercially available starch, the starch microspheres prepared in Example 1, and the starch microspheres prepared in Example 3 were respectively characterized by scanning electron microscopy. The results are shown in FIG. 1. As can be seen, the microspheres prepared by the method in Example 1 have a rough surface, and the microspheres prepared by the method in Example 3 have a smooth surface.

The starch microspheres in the Examples were tested by DSC. The test method is as follows.

Test conditions: One starch sphere prepared in Example 1 or Example 15 or 3.5 mg corn starch was added to a liquid aluminum crucible, and 70% of water was added. The aluminum crucible was sealed, equilibrated in a greenhouse for 24 h, and transferred to a DSC analyzer for heating. Using an empty aluminum tray as a reference, the DSC analyzer was calibrated with indium. The sample tray was heated from 50° C. to 90° C. at a speed of 10° C./min. Parameters such as the initial temperature (To) and the final temperature (Tc) were automatically calculated.

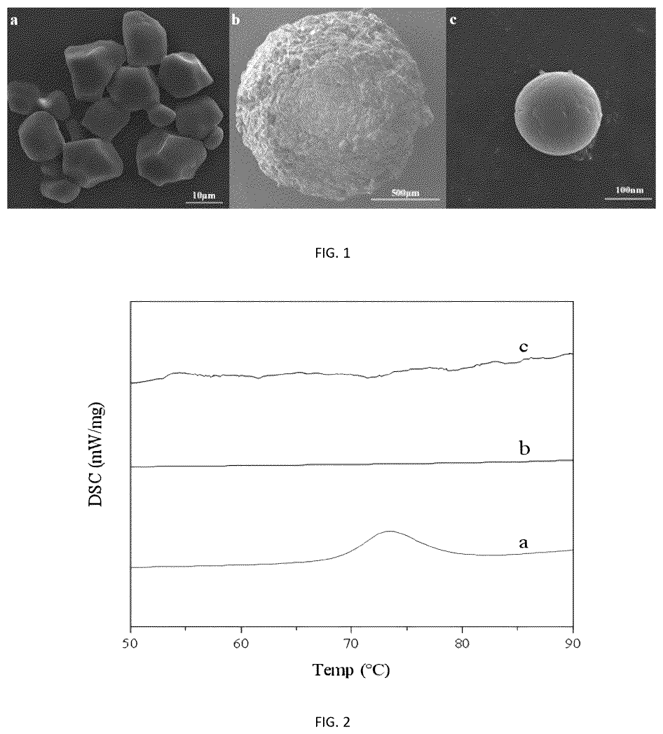

As can be clearly observed from FIG. 2 that the initial gelatinization temperature of the corn starch was 66.9° C. and the final temperature was 76.2° C. Generally, the gelatinization temperature of starch is within a temperature range (initial temperature-final temperature). However, the DSC curves of the starch microspheres prepared in Example 1 and Example 15 during DSC testing did not show an obvious exothermic peak and a gelatinization temperature range, and were not accompanied by a change in energy, indicating that the structure of the starchmicrospheres was in an amorphous state. This was because the heating process during the preparation of the starch microspheres in Example 1 and Example 15 had caused gelatinization of the starch, and the molecular arrangement inside the starch granules had been destroyed. Although the subsequent cooling could cause retrogradation and reassociation of the starch molecules, the starch could not be completely restored to the structural state of raw starch.

The XRD testing method is as follows.

A right amount of starch microspheres or starch was placed in a groove of a sample plate. The surface of the sample was smoothed such that it was flush with the outer surface of the sample plate. Then, the sample plate was placed in an X-ray powder diffractometer. The sample was scanned with Cu-Ka radiation under a tube voltage of 40 kV at a current of 30 mA and a speed of 10°/min. The X-ray powder diffraction patterns were recorded at 20 of 4-70°.

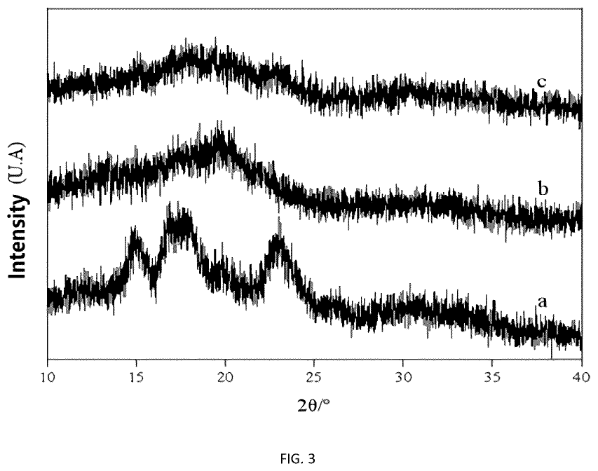

As can be seen from FIG. 3, the corn starch exhibited strong diffraction peaks at 2θ=15.1°, 16.9°, 17.9° and 22.5° and had a crystallinity of about 26.34%, exhibiting a typical A-type crystal structure. The diffraction peaks of the starch microspheres prepared in Example 1 and Example 15 disappeared at 20=15.1, 16.9, 17.9 and 22.5, and the amorphous regions widened, showing a weak V-type crystal structure, which was basically an amorphous structure. The main reason for these changes was that the molecules of raw starch were closely arranged by a large number of intermolecular hydrogen bonds, forming micelles with small gaps therebetween, so the raw starch was insoluble in cold water. During the high-temperature gelatinization process, a part of the micelles was dissolved such that gaps were formed. In this case, water molecules intruded into the gaps between the starch micelles. The water molecules connect to a part of the starch molecules by hydrogen bonds. As a result, the hydrogen bonds of the starch molecules were destroyed, and the molecular structure extended, so that the crystallization ability of the starch microspheres prepared in Example 1 and Example 15 was reduced, resulting in a significant reduction in the crystallinity. Compared with Example 15, the starch microspheres prepared in Example 1 exhibited a diffraction peak at 19.8°, which was because the gelatinized starch undergone retrogradation after being cooled and the starch molecules were re-associated. The diffraction peak of Example 15 disappeared at 19.8°. This may be because the vitamin C added during the preparation of Example 15 made the gelatinization solution acidic, which prevented the retrogradation of gelatinized starch. In this case, the starch molecules could not move and approach to each other, so the micelle structure could not be formed.

Finally, it should be noted that the above contents are merely used to illustrate the technical solutions of the present disclosure and are not intended to limit the protection scope of the present disclosure. Simple modifications or equivalent substitutions of the technical solutions of the present disclosure made by those of ordinary skill in the technology shall not depart from the essence and scope of the technical solutions of the present disclosure.

What is claimed is:

1. A method for preparing natural starch microspheres with a uniform particle size, comprising the following steps:
S1: heating a mixture of natural starch and water for gelatinization to form a water phase;
S2: controlling a temperature of a substance comprising a low-polarity solvent to form an oil phase;
S3: dispersing the water phase obtained in step S1 into the oil phase obtained in step S2 to obtain a mixed solution;
S4: treating the mixed solution obtained in step S3 under reduced pressure, and separation to obtain preliminarily solidified starch microspheres; and
S5: washing to remove the residual oil phase on the surface of the preliminarily solidified starch microspheres, and drying to obtain the natural starch microspheres.

2. The method according to claim 1, wherein the natural starch in step S1 is at least one of corn starch, waxy corn starch, bean starch, potato starch, sweet potato starch, wheat starch, cassava starch, grain amaranth starch, rice starch, water caltrop starch, lotus root starch, kudzu root starch and water chestnut starch.

3. The method according to claim 1, wherein the starch and the water in step S1 are respectively used in an amount of 1 part by mass and 6-100 parts by mass; and the oil phase in step S2 contains an oil phase surfactant.

4. The method according to claim 3, wherein the starch and the water in step S1 are respectively used in an amount of 1 part by mass and 9-50 parts by mass; and the surfactant in step S2 is selected from one of Tween 80 and Span 80, and accounts for 0.1-2% by mass of the oil phase.

5. The method according to claim 1, wherein the gelatinization in step S1 is carried out at a temperature of 50-100° C. for 1-300 min; and the low-polarity solvent in step S2 is at least one of aliphatic hydrocarbon, vegetable oil, animal oil, beeswax and medium chain triglyceride.

6. The method according to claim 5, wherein the gelatinization in step S1 is carried out at a temperature of 75-100° C. for 3-30 min; and the low-polarity solvent in step S2 is one of aliphatic hydrocarbon, beeswax and medium chain triglyceride.

7. The method according to claim 6, wherein the gelatinization in step S1 is carried out at a temperature of 75-80° C. for 3-5 min; and the low-polarity solvent in step S2 is medium chain triglyceride.

8. The method according to claim 1, wherein the temperature in step S2 is 0-100° C.; and further, the temperature in step S2 is 30-80° C.

9. The method according to claim 1, wherein the dispersion in step S3 is at least one of a manual dropping process, a membrane emulsification process, a high-voltage electrostatic control process and a microfluidic process.

10. The method according to claim 9, wherein the dispersion in step S3 is a manual dropping process, and specifically comprises the following steps:
The water phase obtained in step S1 is manually dropped into the oil phase obtained in step S2 with a syringe, and the water phase is dropped in different positions to form droplets that do not adhere to each other, thereby obtaining the mixed system; wherein a needle of the syringe has an inner diameter of 0.1-1 mm, and the oil phase is not stirred during the dropping.

11. The method according to claim 10, wherein the water phase obtained in step S1 has a viscosity of 100-1000 cP.

12. The method according to claim 9, wherein the dispersion in step S3 is a membrane emulsification process, and specifically comprises the following steps:
the water phase obtained in step S1 is passed through a hydrophobic membrane with a pore size of 0.1-50 μm under a transmembrane pressure of 0.01-0.5 MPa, and the filtrate is dispersed into the oil phase obtained in step S2 to obtain the mixed solution.

13. The method according to claim 12, wherein the transmembrane pressure is 0.04-0.4 MPa, and the pore size of the hydrophobic membrane is 0.1-10 μm.

14. The method according to claim 12, wherein the water phase obtained in step S1 has a viscosity of 1.1-10 cP.

15. The method according to claim 1, wherein the reduced pressure treatment in step S4 is carried out at a temperature of 60-80° C. under an atmospheric pressure of 3-50 mmHg for 15-120 min.

16. The method according to claim 1, wherein the drying in step S5 is freeze-drying, and the drying is carried out for 3-12 h.

17. The method according to claim 1, further comprising:
adding at least one of a water-soluble loading object, a water-soluble colloid and a fat-soluble loading object into the water phase in step S1.

18. The method according to claim 17, wherein the water-soluble loading object is selected from at least one of vitamin C, vitamin B2, anthocyanin and folic acid; the water-soluble loading object is used in an amount of 0.01-1 part by mass; the water-soluble colloid is selected from at least one of unhydrolyzed starch octenyl succinate, hydrolyzed starch octenyl succinate, gelatin, gum arabic, sodium carboxymethyl cellulose, sodium lignosulfonate, calcium lignosulfonate, guar gum and xanthan gum; the water-soluble colloid is used in an amount of 0-1 part by mass; the fat-soluble loading object is at least one of vitamin A, vitamin D, vitamin E, paclitaxel and carotinoids; and the fat-soluble loading object is used in an amount of 0.01-1 part by mass.
